(19) United States
(12) Patent Application Publication
(10) Pub. No.: US 2008/0188911 A1
(43) Pub. Date: Aug. 7, 2008

(54) Hot compress apparatus

(76) Inventor: Mei-Feng Chao, Yung Ho City (TW)

Correspondence Address: PRO-TECHTOR INTERNATIONAL SERVICES, 20775 NORADA CT., SARATOGA, CA, 95070, US

(21) Appl. No.: 11/704,353

(22) Filed: Feb. 7, 2007

(51) Int. Cl. A61F 7/02

(52) U.S. Cl. 607/96

(57) ABSTRACT

A hot compress apparatus that is used on the human body for providing safe and comfortable hot compress treatment and shock stimulation. The hot compress apparatus comprises at least: a flexible base, a circuit device, a heat conductive member disposed on the base and electrically connected to the circuit device for receiving electrical heat and electric power from a main controller, so as to enable the main controller to adjust the electric power transmitted to the heat conductive member, ensuring that hot compress temperature is at a precise, comfortable and safe condition.

### BACKGROUND OF THE INVENTION

1. Field of the Invention

The present invention relates to a hot compress apparatus, and more particularly to a hot compress apparatus that is used on the human body for providing safe and comfortable hot compress treatment and shock stimulation.

2. Description of the Prior Art

Hot compress has been more recognized and widely practiced in medical circles, the heat energy of the hot compress can promote blood circulation, improving metabolism, and has a preventive effect.

U.S. Pat. No. 6,302,901B1 discloses an apparatus for thermal low periodic wave treatment that comprises a main control circuit with a CPU, and the apparatus can provide thermal shock and low periodic waves for stimulating the muscles to cause a smooth blood circulation. The low periodic waves can control the output strength and the output time. When cooperating with a multiple-stage temperature control, the temperature and the strength of the low periodic wave pulse can be selected (in multiple stages) via PWM control. The PWM control, as a means for driving the low periodic wave type elevation circuit, can effectively improve the electric power efficiency. Furthermore, a feedback monitoring circuit serves to reset the pole strength, preventing the output of an inappropriate voltage. The apparatus comprises a pair of shock plates, a storage battery and a main control circuit. The main control circuit includes a single chip CPU, and a DC charger switching circuit directly connected thereto, a voltage-stabilizing circuit, a ringing circuit, a heating circuit, a press key control circuit, a voltage-elevating and feedback monitoring circuit, a polar switching circuit and a display. The shock plates are adhered directly to the human body for outputting a heat source and low periodic wave pulse source, thus providing an effect of promoting the blood circulation and improving metabolism.

However, it is uncomfortable and unsafe when the shock plates are in direct contact with the human body.

The present invention has arisen to mitigate and/or obviate the afore-described disadvantages.

### SUMMARY OF THE INVENTION

The primary objective of the present invention is to provide a hot compress apparatus that is used on the human body for providing safe and comfortable hot compress treatment and shock stimulation. The hot compress apparatus comprises at least: a flexible base, a circuit device, a heat conductive member disposed on the base and electrically connected to the circuit device for receiving electrical heat and electric power from the main controller, so as to enable the main controller to adjust the electric power transmitted to the heat conductive member, ensuring that hot compress temperature is at a precise, comfortable and safe condition.

The circuit device can be installed on the base directly.

The base further includes at least one sensor for sensing real status, such temperature status, and then status data is transmitted to the circuit device and then is to be processed by a main controller.

The base includes at least one shock plate electrically connected to the circuit device for transmitting the electric power to the human body.

The hot compress apparatus further comprises a coating structure for covering the base and the circuit device.

### BRIEF DESCRIPTION OF THE DRAWINGS

FIG. 1 is an exploded view of showing a hot compress apparatus in accordance with a preferred embodiment of the present invention;

FIG. 2 is an assembly view of showing the hot compress apparatus in accordance with the preferred embodiment of the present invention;

FIG. 3 is an exploded view of showing a hot compress assembly in accordance with a preferred embodiment of the present invention;

FIG. 4 is an assembly view of showing the hot compress assembly in accordance with a preferred embodiment of the present invention;

FIG. 5 is another assembly view of showing the hot compress assembly in accordance with a preferred embodiment of the present invention; and

FIG. 6 is an enlarged illustration view of showing A section of FIG. 1.

### DETAILED DESCRIPTION OF THE PREFERRED EMBODIMENT

The foregoing, and additional objects, features and advantages of the present invention will become apparent from the following detailed description of preferred embodiments thereof, taken in conjunction with the accompanying drawings.

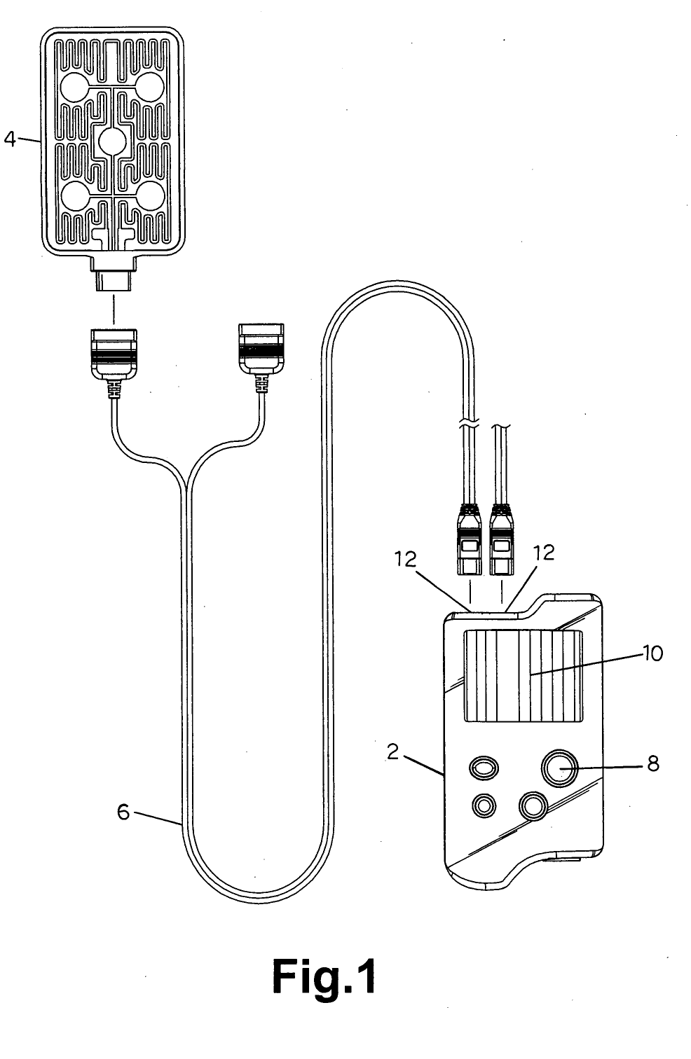

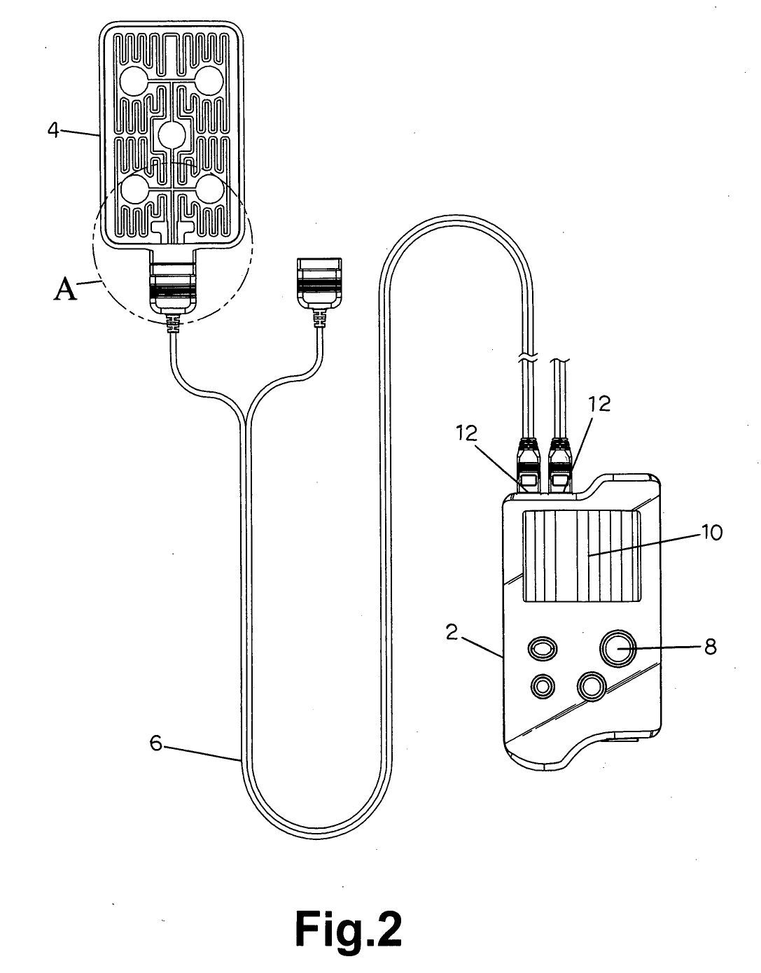

Referring to FIGS. 1 and 2, a hot compress apparatus in accordance with a preferred embodiment of the present invention comprises a main controller 2 and at least one hot compress assembly 4. The hot compress assembly 4 is electrically connected to a transmission line 6. The main controller 2 includes a control panel 8, a display 10, and at least one connecting seat 12. The control panel 8 controls the mode of use, and the signal regarding the mode of use is outputted via the connecting seat 12. The operation condition of the control panel 8 is displayed on the display 10 for the user' reference.

The main controller 2 is connected to the transmission line 6 via the connecting seat 12, so that the electric power and the control signal of the main controller 2 are transmitted to the hot compress assembly 4 by the transmission line 6, instructing the hot compress assembly 4 to carry out the predetermined work. Such as: hot compress and/or electrically shock treatment.

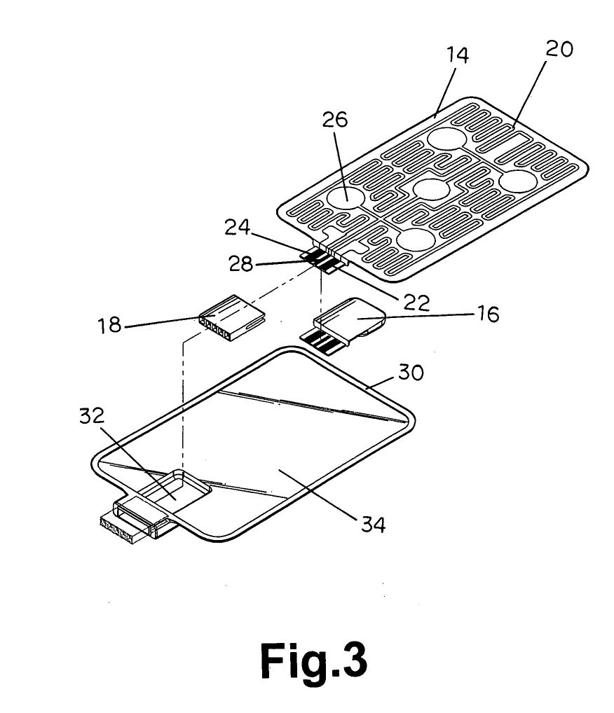

The hot compress assembly 4, with reference to FIGS. 3-6, includes:

A flexible base 14 can be formed into any shape.

A circuit device 16 can be arranged on the base 14 directly, or can be a device separated from the base 14, such as a circuit board. The circuit board has a connecting terminal 18 that can be in the form of a plurality of connecting points (not shown) or a connector electrically connected to the transmission line 6. The connecting terminal 18 and the circuit device 16 can also be separately arranged.

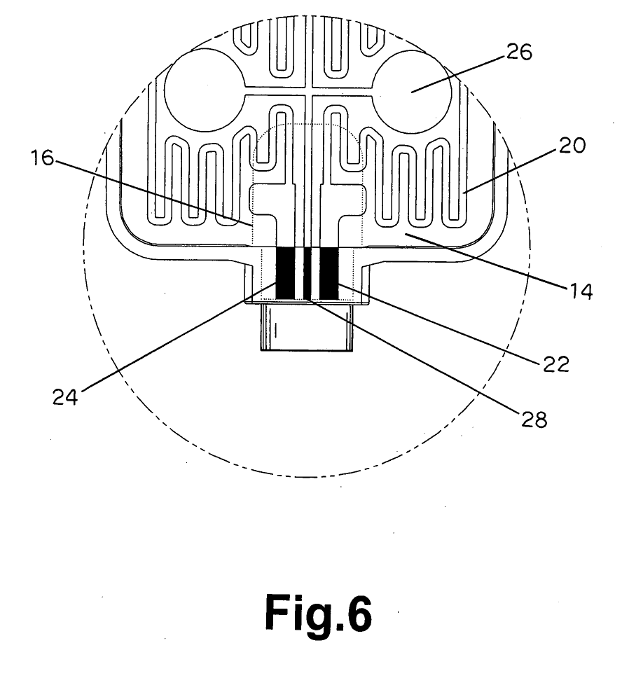

A heat conductive member 20 is installed on the base 14, for example it is averagely arranged on the base 14 in a zigzag pattern, so as to enable the heat generated by the heat conductive member 20 to be evenly distributed on the base 14 for providing hot compress on the surface of the human body. The heat conductive member 20 has a first terminal 22 and a second terminal 24 that are to be electrically connected to the aforementioned circuit device 16 (as shown in FIGS. 5 and 6).

At least one shock plate 26 is disposed on the base 14 and can be an electrical treatment shock plate. The shock plate 26 has a third terminal 28 to be electrically connected to the circuit device 16 for transmitting the electric power to the human body.

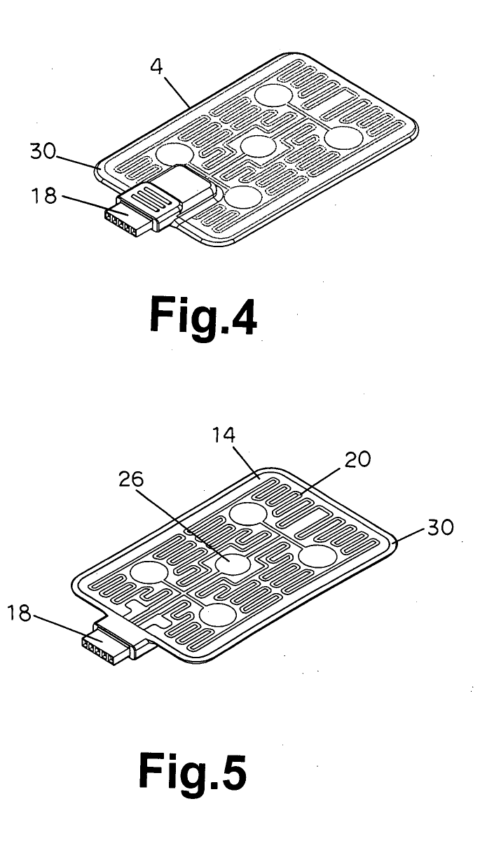

A coating structure 30 includes a receiving space 32 and a coating surface 34. The receiving space 32 is provided for accommodation of the circuit device 16. The circuit device 16 is electrically connected to the first and second terminals 22, 24 of the heat conductive member 20 and to the third terminal 28 of the shock plate 26. The coating surface 34 has a gluing surface covered with a release film, after removing the film, the base 14 and the circuit device 16 can be glued to the gluing surface of the coating surface 34 (as shown in FIGS. 4 and 5). However, the coating structure 30 doesn't cover the shock plate 26, namely, the shock plate 26 is exposed out of the coating structure 30. In this way, the electric power of the main controller 2 can be transmitted to the human body. The coating structure 30 ensures a good hot compress effect, providing an even-distributed heat and having an isolation effect.

The gluing surface can also be formed on the base 14 (not shown) for gluing the coating structure 30, and in this way, the coating structure 30 can also be glued to the base 14 for covering the base 14 and the circuit device 16.

In addition, the base 14 can further includes at least one sensor (not shown) for sensing the real status: such as temperature status. And then the status data is transmitted to the circuit device 16 and then is processed by the main controller 2, so as to enable the main controller 2 to adjust the electric power transmitted to the heat conductive member 20, ensuring that hot compress temperature is at a precise, comfortable and safe condition. In the use of one hot compress assembly 4, the hot compress assembly 4 can be provided with heat conductive member 20 and at least one shock plate 26. If only one shock plate 26 is exposed out of the base 14, and two hot compress assemblies 4 are needed to stick to the human body. At this moment, the shock plates 26 of the two hot compress assemblies 4 and the human body form an electric circuit. When any of the hot compress assemblies 4 drops off the human body, it can determine the circuit is broken or not by judging if the shock plates 26 of the two hot compress assemblies 4 and the human body form a complete electric circuit, providing a function of cut-off protection. Or, it can provide at least two shock plates 26 on a single hot compress assembly 4, when the hot compress assembly 4 drops off the human body, it can determine the circuit is broken or not by judging if the shock plates 26 and the human body form a complete electric circuit, providing a function of cut-off protection.

Since the abovementioned shock plates 26 form a complete circuit by cooperating with the human body, when the electric power outputted from the main controller 2 is transmitted to the shock plates 26, the hot compress assembly 4 will produce electrical shock stimulation.

In addition, the aforementioned transmission line 6 is electrically connected to the circuit device 16 and serves to transmit electric power and data.

The main controller 2 can also be arranged on the circuit device 16.

While we have shown and described various embodiments in accordance with the present invention, it is clear to those skilled in the art that further embodiments may be made without departing from the scope of the present invention.

What is claimed is:

1. A hot compress apparatus comprising at least:
a flexible base;
a circuit device; and
a heat conductive member disposed on the base and electrically connected to the circuit device.

2. The hot compress apparatus as claimed in claim 1, wherein the circuit device is installed on the base directly.

3. The hot compress apparatus as claimed in claim 1, wherein the circuit device is a circuit board separated from the base.

4. The hot compress apparatus as claimed in claim 1, wherein the base includes at least one shock plate electrically connected to the circuit device.

5. The hot compress apparatus as claimed in claim 1 further comprising a coating structure for covering and fixing the base and the circuit device.

6. The hot compress apparatus as claimed in claim 5, wherein the coating structure includes a receiving space for accommodation of the circuit device, a gluing surface is formed on the base for contacting the coating structure, so as to fix the coating structure on the base.

7. The hot compress apparatus as claimed in claim 1, wherein the base further includes at least one sensor for sensing real status, and then status data is transmitted to the circuit device and then is to be processed by a main controller.

8. The hot compress apparatus as claimed in claim 1 further comprising a transmission line electrically connected to the circuit device for transmitting date and electric power.

9. A hot compress apparatus comprising a main controller, the main controller being electrically connected to at least one hot compress assembly, the main controller including a control panel for controlling mode of use, and a display for displaying operating status of the control panel; the at least one hot compress assembly including at least:
a flexible base,
a circuit device disposed on the base and having a connecting terminal to be electrically connected to the main controller; and
a heat conductive member disposed on the base and electrically connected to the circuit device for receiving electrical heat and electric power from the main controller.

10. The hot compress apparatus as claimed in claim 9, wherein the circuit device is installed on the base directly.

11. The hot compress apparatus as claimed in claim 9, wherein the circuit device is a circuit board separated from the base.

12. The hot compress apparatus as claimed in claim 9, wherein the base includes at least one shock plate electrically connected to the circuit device.

13. The hot compress apparatus as claimed in claim 9 further comprising a coating structure for covering and fixing the base and the circuit device.

14. The hot compress apparatus as claimed in claim 13, wherein the coating structure includes a receiving space for accommodation of the circuit device, a gluing surface is formed on the base for contacting the coating structure, so as to fix the coating structure on the base.

15. The hot compress apparatus as claimed in claim 9, wherein the base further includes at least one sensor for sensing real status, and then status data is transmitted to the circuit device and then is to be processed by the main controller.
